(19) United States
(12) Patent Application Publication
(10) Pub. No.: US 2008/0112844 A1
(43) Pub. Date: May 15, 2008

(54) PHOTOCATALYTIC AIR PURIFYING DEVICE

(75) Inventor: John R. Garrett, Vero Beach, FL (US)

Correspondence Address: DOVAS LAW P.C., 307 BAINBRIDGE STREET, PHILADELPHIA, PA, 19147, US

(73) Assignee: AIRGORILLA CORPORATION, Vero Beach, FL (US)

(21) Appl. No.: 11/936,794

(22) Filed: Nov. 8, 2007

Related U.S. Application Data
(60) Provisional application No. 60/865,038, filed on Nov. 9, 2006.

(51) Int. Cl. A61L 9/20; G05B 23/00

(52) U.S. Cl. 422/4; 422/119; 422/122

(57) ABSTRACT

An air purifying device including a mounting body configured for connection to a duct or a plenum of an air handling system is provided. A low-profile porous substrate including a photocatalyst is connected to and extends from the mounting body. An ultraviolet lamp is connected to and extends from the mounting body adjacent to the low-profile porous substrate for activating the photocatalyst. The present invention further provides methods of purifying air within an air handling system.

### CROSS REFERENCE TO RELATED APPLICATION(S)

This application claims the benefit of U.S. provisional application No. 60/865,038, filed Nov. 9, 2006, which is incorporated by reference as if fully set forth.

### BACKGROUND

The air within our living and working environments may include several types of harmful contaminates including particles, organic and inorganic gases, and bioaerosols such as bacteria, viruses and molds. Photocatalytic air purifiers may be used to remove such contaminates from the air. Such devices typically use an ultraviolet light for radiating a titanium dioxide photocatalyst deposited on a substrate to create oxidizers capable of reacting with contaminates in air passing over the substrate to convert the contaminates into less harmful or completely benign substances.

Known photocatalytic air purifiers may be implemented in an enclosed environment to provide effective air purification to promote the health of occupants therein. However, known devices lack the level of compatibility required for them to be easily and effectively integrated with existing air handling systems such as a residential or commercial HVAC systems, or provide insignificant amounts of photocatalytic material for effective air purification. Accordingly, those desiring the benefits of effective photocatalytic air purification must resort to stand alone systems which may be obstructive, or alternatively, incur significant expense in redesigning and rebuilding an existing air handling system to achieve compatibility with a known device.

In view of the above, it would be desirable to provide a photocatalytic air purifying device which can be easily integrated with existing air handling systems to provide effective air purification. Such system should further be easily integrated with new air handling systems using conventional methods of air handling system design.

### SUMMARY

The present invention provides an air purifying device including a mounting body configured for connection to a duct or a plenum of an air handling system. A low-profile porous substrate including a photocatalyst is connected to and extends from the mounting body. An ultraviolet lamp is connected to and extends from the mounting body adjacent to the low-profile porous substrate for activating the photocatalyst.

The present invention also provides a method of purifying air within an air handling system. The method includes providing an air purifying device with a mounting body, a low-profile porous substrate with a photocatalyst connected to and extending from the mounting body, and an ultraviolet lamp connected to and extending from the mounting body adjacent to the low-profile porous substrate. The low-profile porous substrate is positioned within an air flow path of the air handling system, and the photocatalyst is activated with the ultraviolet lamp.

The present invention further provides another method of purifying air within an air handling system. This method includes providing a low-profile porous substrate including a photocatalyst and providing an ultraviolet lamp for activating the photocatalyst. The low-profile porous substrate is inserted approximately perpendicular to a flow path of the air handling system. The ultraviolet lamp is inserted adjacent to the low-profile porous substrate within the flow path of the air handling system, and the substrate is irradiated with the ultraviolet lamp to activate the photocatalyst.

### BRIEF DESCRIPTION OF THE DRAWING(S)

The foregoing Summary as well as the following detailed description will be readily understood in conjunction with the appended drawings which illustrate preferred embodiments of the invention. In the drawings:

FIG. 1 is top front perspective view of a photocatalytic air purifying device according to a preferred embodiment of the present invention.

FIG. 2 is a front perspective view of the photocatalytic air purifying device of FIG. 1.

FIG. 3 is right rear perspective view of the photocatalytic air purifying device of FIG. 1.

FIG. 4 is a left front perspective view of the photocatalytic air purifying device of FIG. 1.

FIG. 5 is a section view of a portion of an air duct in which the photocatalytic air purifying device of FIG. 1 is installed, showing a rear elevation view of the photocatalytic air purifying device.

FIG. 6 is a diagram showing a method of purifying air within an air handling system according to a preferred embodiment of the present invention.

FIG. 7 is a diagram showing another method of purifying air within an air handling system according to a preferred embodiment of the present invention.

FIG. 8 is a rear perspective view of a photocatalytic air purifying device according to another preferred embodiment of the present invention.

FIG. 9 is a front perspective view of the photocatalytic air purifying device of FIG. 8.

### DETAILED DESCRIPTION OF THE PREFERRED EMBODIMENT(S)

Certain terminology is used in the following description for convenience only and is not limiting. The words “right,” “left,” “top,” and “bottom” designate directions in the drawings to which reference is made. The words “a” and “one” are defined as including one or more of the referenced item unless specifically stated otherwise. This terminology includes the words above specifically mentioned, derivatives thereof, and words of similar import. The phrase “at least one” followed by a list of two or more items, such as A, B, or C, means any individual one of A, B or C as well as any combination thereof.

The preferred embodiments of the present invention are described below with reference to the drawing figures where like numerals represent like elements throughout.

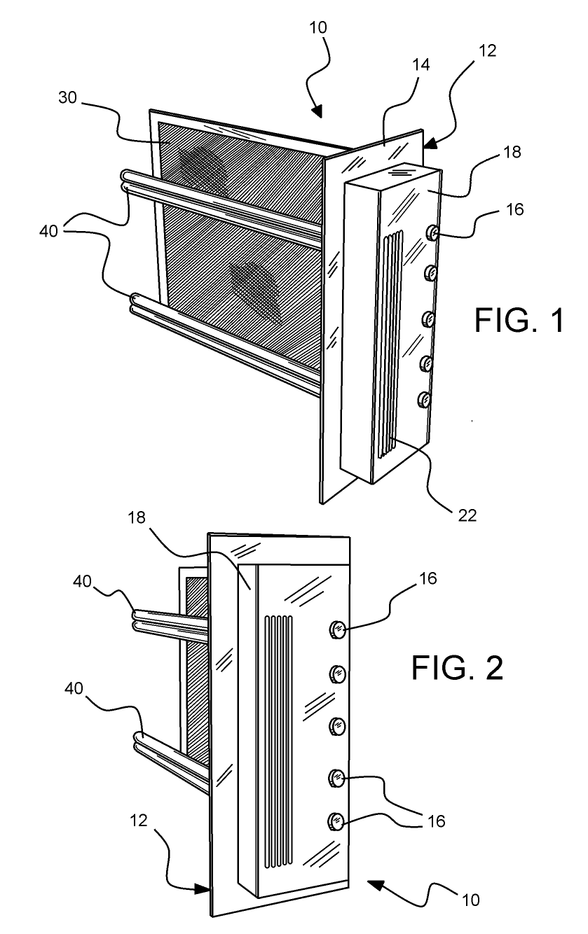

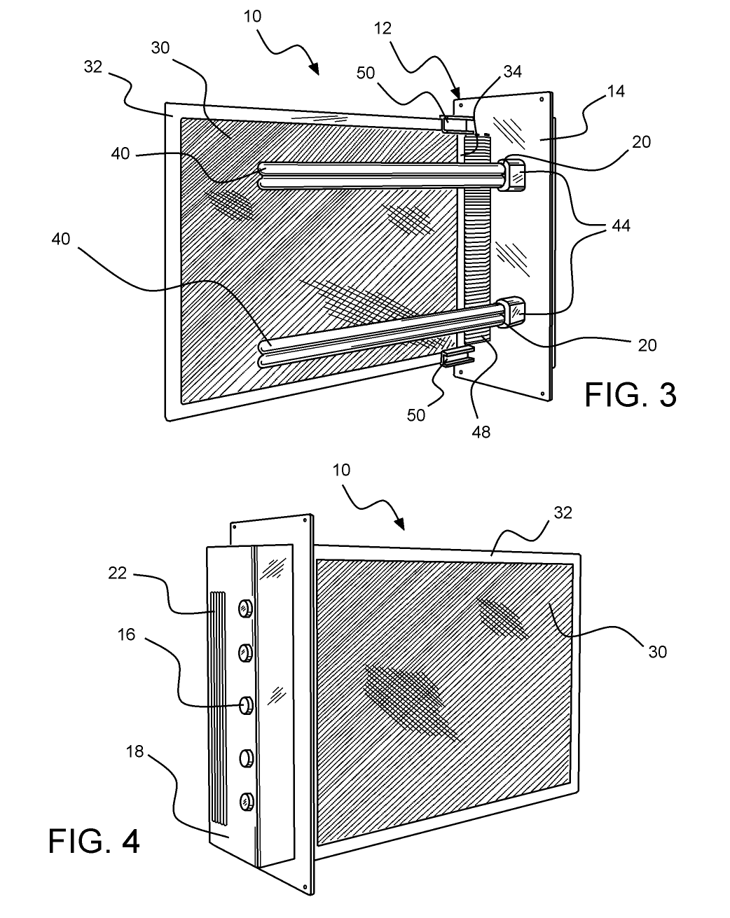

Referring to FIGS. 1-5, an air purifying device 10 according to a preferred embodiment of the present invention is shown. The air purifying device 10 includes a mounting body 12 configured for connection to a duct or a plenum of an air handling system such as a residential or commercial HVAC system. A low-profile porous substrate 30 including a photocatalyst is connected to and extends from the mounting body 12. Ultraviolet lamps 40 are also connected to and extend from the mounting body 12 adjacent to the low-profile porous substrate 30 for activating its photocatalyst. A plurality of indicator lights 16, which are preferably LEDs, are provided on a removable cover 18 of the mounting body 12 for indicating a status of the air purifying device 10 including a power on/off and ultraviolet lamp on/off status.

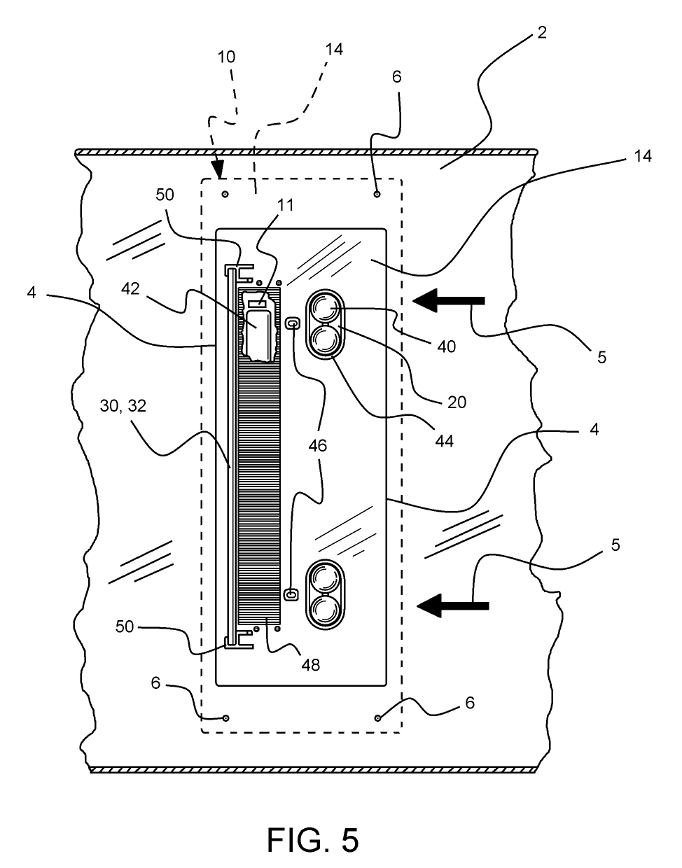

The mounting body 12 includes a mounting plate 14 configured for connection to a surface of a duct or a plenum of an air handling system, for example the duct 2 shown in FIG. 5. The mounting plate 14 may include mounting holes to facilitate attachment to a duct or plenum of an air handling system, for example via screws 6. While the mounting plate 14 is shown connected to directly to the duct 2 with the substrate 30 and the ultraviolet lamps 40 extending through an aperture 4 therein, alternatively, the mounting plate 14 can be connected to a housing for connection in any suitable manner to an air handling system within an air flow path.

As shown, the low-profile porous substrate 30 is rectangular, substantially flat and includes a planar surface which preferably includes one or more of a perforated plate, grid, screen, mesh or filter to allow through passage of a flow of air. A frame 32 may be provided to support the substrate 30 as shown. The porous substrate 30 preferably includes a large surface area as a result of surface unevenness, irregularities or the like, such that a substantial amount of photocatalyst may be exposed. The substrate 30 extends, as shown, substantially perpendicular from the surface of the mounting plate 14, and is held in place by mounting tracks 50 which are permanently affixed to the mounting plate 14. Alternatively, the substrate 30 may extend approximately perpendicularly from the surface of the mounting plate 14, for example at any suitable angle between about 70 degrees and 110 degrees from the mounting plate 14. Alternatively, the low-profile porous substrate 30 may have an arced or bent form for example to provide structural rigidity or desired air flow properties within a duct or plenum, and further may be configured as another suitable shape other than rectangular, for example circular or irregularly shaped.

Preferably, a height of the substrate 30, defined by an edge 34 of the substrate 30 adjacent to the mounting plate 14, is at least 75 percent a length of the mounting plate, such that in an installed position in a section of a flow path of a duct or plenum, the substrate 30 encompasses a significant portion of the flow area of the flow path in the section. Preferably, the substrate 30 through its porosity is configured to provide ample air through flow to minimize pressure drop when installed in a duct or plenum of an air handling system.

The substrate 30 is preferably constructed of one or more suitable porous materials capable of receiving or being formed integral with the photocatalyst. The substrate 30 is firmly connected to the mounting body 12 to prevent breakage from handling or from the passage of high speed air flow. A frame 32 is preferably provided to support the substrate 30 and to engage the mounting tracks 50, as shown, allowing for less rigid materials, such as mesh or screen materials of metallic or polymeric construction, to be used in forming the substrate 30. The photocatalyst preferably includes titanium dioxide which is coated onto the substrate 30. Alternatively, any photocatalyst suitable for activation by an ultraviolet light source may be applied to the substrate 30 or otherwise formed integrally with the substrate 30.

The ultraviolet lamps 40 are preferably, as shown, elongated and positioned adjacent to the planar surface of the low-profile porous substrate 30 substantially, or alternatively approximately, perpendicular to a surface of the mounting plate 14 for activating the photocatalyst by ultraviolet irradiation. The ultraviolet lamps 40 are preferably suitably spaced from the low-profile porous substrate 30 to allow sufficient irradiation of the entire surface thereof. The ultraviolet lamps 40 preferably emit relatively intense radiation at a wavelength of about 254 nm, which corresponds to the wavelength at which ozone is photolysed resulting in oxygen radicals and excited oxygen. More preferably, the ultraviolet lamps 40 emit relatively intense radiation across a broad spectrum of wavelengths between 200 nm and 300 nm. Alternatively, the ultraviolet lamps 40 may further emit radiation at ozone-generating wavelengths including wavelengths less than 200 nm, such as an ideal ozone-generating wavelength of about 185 nanometers. As shown in FIG. 5, the ultraviolet lamps 40 are preferably provided upstream from the substrate 30 when installed in an air flow path of an air handling system, the direction of air flow being indicated by the arrows 5.

A ballast 42 is preferably provided on a surface of the mounting body 12 under the cover 18 for starting the ultraviolet lamps 40 and regulating current thereto. Alternatively, two or more ballasts or other suitable power supply may be provided for powering the lamps 40, and further the ballast 42 or other suitable power supply may be provided in a separate and/or remote enclosure to facilitate its removal and replacement. Preferably, a power supply is also provided for powering the indicator lights 16, and alternatively, if the ballast 42 is not provided, such power supply may be configured for powering the ultraviolet lamps 40. Preferably, air vents 22 are provided in the cover 18 to promote cooling of the ballast 42 and other electronic components through air flow. A finned heat sink 48 is also provided attached to the mounting plate 14 for cooling the ballast 42 and other electronic components.

A timing device is preferably provided connected to the ultraviolet lamps 40 and one or more of the indicator lights 16. The timing device is configured to measure the amount of time the ultraviolet lamps 40 have been lit and signal when one or both of the ultraviolet lamps 40 have been powered for a predetermined time period to indicate, preferably using one or more of the indicator lights 16, when a useful life of one or both of the lamps 40 has expired. Preferably, in addition to or as an alternative to the timing device, radiation sensors 46 are provided for measuring the ultraviolet light intensity of the lamps 40, the sensors 46 being configured to signal one or more of the indicator lights 16 when a prescribed diminished light intensity is reached indicating that a useful life of one or both of the lamps 40 has expired. A processor 11, preferably including the timing device, is connected to the sensors 46 and system power and controls the indicator lights 16 to indicate a status of the air purifying device 10.

The cover 18 of the mounting body conceals the control components of the lamps 40 including the ballast 42, the processor 11 and electronic circuitry supporting the indicator lights 16. The air purifying device 10 is preferably configured to accept line electric power, for example standard 110V or 220V AC line power.

The ultraviolet lamps 40 are preferably of sturdy design and firmly connected to the mounting body 12 by the protuberances 44 to prevent breakage from handling or from the passage of high speed air flow. Thru apertures 20 are formed in protuberances 44 to permit removal and replacement of the ultraviolet lamps 40 through an outside portion of the mounting body 12, opposite the direction of extension of the ultraviolet lamps 40, after removal of the cover 18, such that the air purifying device 10 does not require removal from its installed position in an air handling system when lamp replacement is required. Alternatively, the mounting body 12 may be provided without the thru apertures 20, and the lamps 40 may be affixed to the mounting body 12 by substantially permanently secured flanges or lamp sockets such that removal of the mounting body 12 from a duct or plenum to which it was installed would be necessary to replace the lamps 40.

While not wishing to be bound by any particular theory of functionality, the elongated shape of the ultraviolet lamps 40 and their position in proximity to the substrate 30 permits superior activation of the photocatalyst on the substrate 30, creating oxygen-containing radicals for oxidizing harmful contaminates, without significantly hindering air flow through an air handling system in which the air purifying device 10 is installed. Its relatively large surface area permits the substrate 30 to encompass a significant portion of a flow area of a flow path in a section in which it is installed, resulting in effective treatment of air passing there through. Further, since two ultraviolet lamps 40 are provided, the substrate 30 is effectively radiated from multiple directions, decreasing the amount of photocatalyst which is non-activated due to surface shadows on the substrate 30, for example surface shadows caused by surface roughness of a mesh substrate. Moreover, the ultraviolet radiation is emitted by the ultraviolet lamps 40 in all directions around the lamps 40, including upstream and downstream of a flow path in which the air purifying device 10 is installed. As such, the ultraviolet lamps 40 alone provide a level of germicidal disinfection of a passing airflow, complementing the oxidization of harmful contaminates stimulated by the photocatalyst, especially in the case where the ultraviolet lamps 40 are configured for emitting a broad spectrum of wavelengths between 200 nm and 300 nm suitable for disinfection and germicidal functionality.

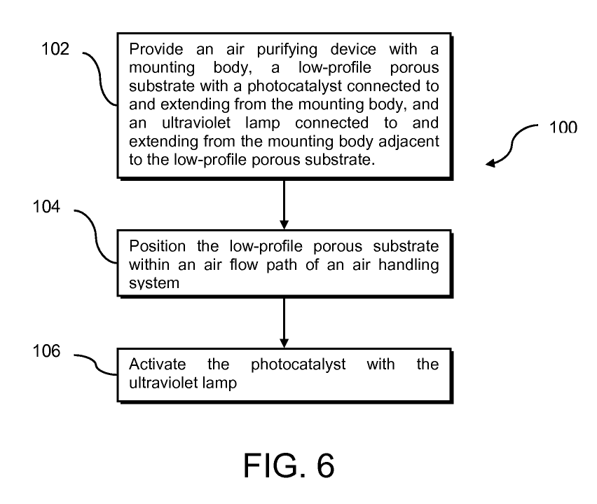

Referring to FIG. 6, a diagram of a method 100 of purifying air within an air handling system according to a preferred embodiment of the present invention is shown. The method includes providing an air purifying device with a mounting body, a low-profile porous substrate with a photocatalyst connected to and extending from the mounting body, and an ultraviolet lamp connected to and extending from the mounting body adjacent to the low-profile porous substrate (step 102). The low-profile porous substrate is positioned within an air flow path of the air handling system (step 104), and the photocatalyst is activated with the ultraviolet lamp (step 106).

Preferably, the method includes providing the ultraviolet lamp as an elongated ultraviolet lamp, providing the low-profile porous substrate as substantially flat with a planar surface, and positioning the planar surface and the elongated ultraviolet lamp approximately perpendicular with the flow path of the of the air handling system. The elongated ultraviolet lamp and the low profile porous substrate are preferably inserted through an aperture of a duct or a plenum of the air handling system. The aperture may be preexisting or created onsite, for example to install the air purifying device in a preexisting air handling system. The mounting body may be connected to the duct or plenum using fasteners, tapes, adhesives, or alternatively, in any suitable manner.

Preferably, the air purifying device is positioned in a section of the flow path wherein an area of the low-profile substrate comprises at least approximately 25 percent of the flow area of the flow path in the section. More preferably, the air purifying device is positioned in a section of the flow path wherein an area of the low-profile substrate comprises at least approximately 75 percent of the flow area of the flow path in the section. In such manner, a significant portion of the air flow passing the substrate may pass over and through the substrate resulting in effective air treatment.

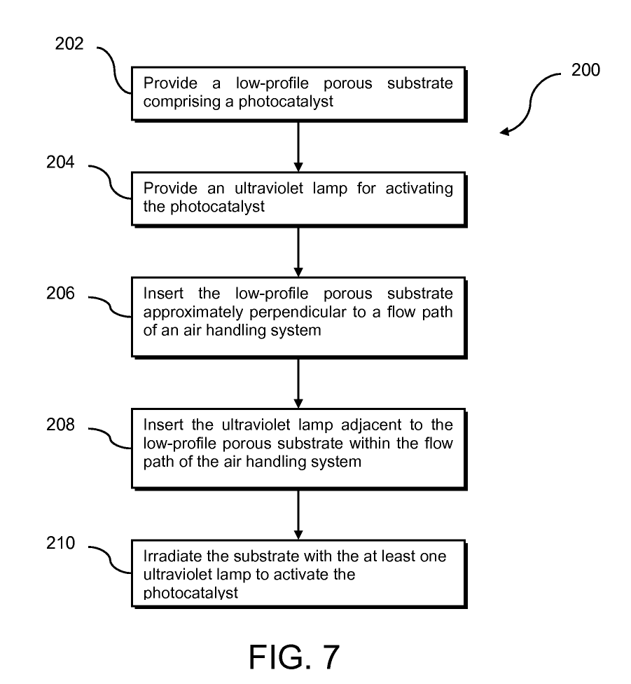

Referring to FIG. 7, a diagram of a method 200 of purifying air within an air handling system according to a preferred embodiment of the present invention is shown. The method 200 includes providing a low-profile porous substrate including a photocatalyst (step 202). An ultraviolet lamp for activating the photocatalyst is provided (step 204). The low-profile porous substrate is inserted approximately perpendicular to a flow path of the air handling system (step 206). The ultraviolet lamp is inserted adjacent to the low-profile porous substrate within the flow path of the air handling system (step 208), and the substrate is irradiated with the ultraviolet lamp to activate the photocatalyst (step 210).

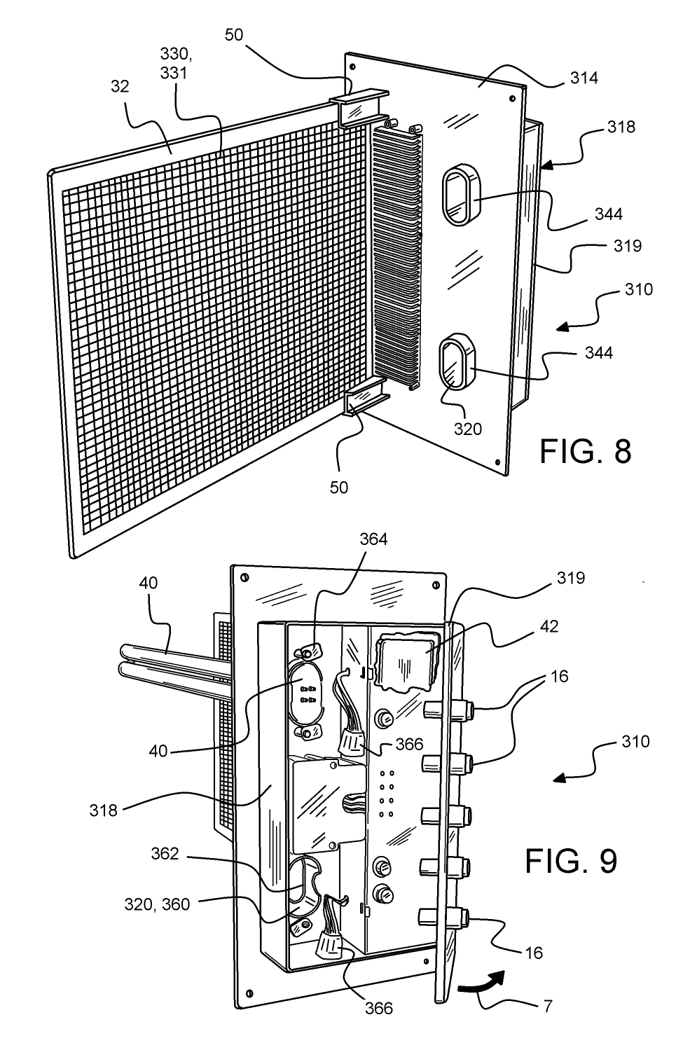

Referring to FIGS. 8 and 9, an air purifying device 310 according to another preferred embodiment of the present invention is shown. The air purifying device 310 is functionally similar to the air purifying device 10 described above. For clarity, FIG. 8 shows the air purifying device 310 without the ultraviolet lamps 40, and FIG. 9 shows the air purifying device 310 with only one ultraviolet lamp 40. The air purifying device 310 includes a cover 318 having a pivoting door 319 which swings open in a direction shown by an arrow 7 to provide access to the ultraviolet lamps 40 and the electronic components including the ballast 42.

Protuberances 344 are fixed to a mounting plate 314 and include an inner portion 360. The protuberances 344 each define an aperture 320 which closely surrounds the ultraviolet lamp 40 to prevent stress-induced failure for example due to loading caused by air flow or handling of the device. The protuberances 344 include a lip 362 and pivoting latches 364 which firmly and removably secure a base of the ultraviolet lamp 40 therebetween. Power from the ballast 42 is provided to the ultraviolet lamps 40 through removable connectors 366.

A substrate 330 preferably includes an outer grid 331 connected to the frame 32, as shown, and a porous mesh material, the porous mesh material being hidden for clarity. The outer grid 331 is preferably provided as two pieces, retaining the porous mesh material therebetween. The substrate 330, including the outer grid 331 and the porous mesh material, may be formed of any suitable metallic, polymeric or composite material and one or both of the outer grid 331 and the porous mesh material are coated with the photocatalyst.

While the preferred embodiments of the invention have been described in detail above, the invention is not limited to the specific embodiments described above, which should be considered as merely exemplary. Further modifications and extensions of the present invention may be developed, and all such modifications are deemed to be within the scope of the present invention as defined by the appended claims.

What is claimed is:

1. An air purifying device comprising:
a mounting body configured for connection to at least one of a duct and a plenum of an air handling system;
a low-profile porous substrate comprising a photocatalyst connected to and extending from the mounting body; and
at least one ultraviolet lamp connected to and extending from the mounting body adjacent to the low-profile porous substrate for activating the photocatalyst.

2. The air purifying device of claim 1, wherein:
the mounting body comprises a mounting plate configured for connection to a surface of the at least one of the duct and the plenum of the air handling system; and
the low-profile porous substrate is substantially flat and comprises a planar surface.

3. The air purifying device of claim 2, wherein the at least one ultraviolet lamp comprises at least two elongated ultraviolet lamps positioned adjacent to the planar surface of the low-profile porous substrate for irradiating the planar surface to activate the photocatalyst.

4. The air purifying device of claim 3, wherein the low-profile porous substrate and the at least two elongated ultraviolet lamps extend approximately perpendicular from a surface of the mounting plate.

5. The air purifying device of claim 3, wherein the mounting plate is configured for positioning the planar surface of the low-profile porous substrate and the at least two elongated ultraviolet lamps approximately perpendicular to a direction of air flow through the at least one of the duct and the plenum.

6. The air purifying device of claim 2, wherein the mounting plate is configured for positioning the planar surface of the low-profile porous substrate approximately perpendicular to a direction of air flow through the at least one of the duct and the plenum.

7. The air purifying device of claim 2, wherein a height of the porous substrate, defined by an edge of the porous substrate adjacent to the mounting plate, is at least 75% a length of the mounting plate.

8. The air purifying device of claim 1, wherein the low-profile porous substrate and the at least one ultraviolet lamp extend approximately perpendicular from a surface of the mounting plate.

9. The air purifying device of claim 1, wherein the photocatalyst comprises a coating of titanium dioxide on the low-profile porous substrate.

10. The air purifying device of claim 1, wherein the at least one ultraviolet lamp emits at least a wavelength of radiation of about 254 nm.

11. The air purifying device of claim 1, further comprising a timing device connected to the at least one ultraviolet lamp configured to signal when the at least one ultraviolet lamp has been powered for a predetermined time period.

12. The air purifying device of claim 1, wherein the at least one ultraviolet lamp is removably connected to the mounting body, wherein the at least one ultraviolet lamp is removable through an aperture in the mounting body from a side of the mounting body approximately opposite the direction of extension of the at least one ultraviolet lamp adjacent to the low-profile porous substrate.

13. The air purifying device of claim 1, further comprising a protuberance connected to and extending from the mounting body, the protuberance including a thru aperture which closely and removably receives the at least one ultraviolet lamp, wherein the protuberance is configured to receive the at least one ultraviolet lamp from at least a side of the mounting body approximately opposite to the direction of extension of the at least one ultraviolet lamp adjacent to the low-profile porous substrate.

14. The air purifying device of claim 1, further comprising:
a processor connected to the at least one ultraviolet lamp and configured for connection to an electric power source; and
a plurality of indicator lights connected to the processor configured to receive signals from the processor indicating a status of the at least one ultraviolet lamp and a status of electric power.

15. A method of purifying air within an air handling system comprising:
providing an air purifying device comprising:
a mounting body;
a low-profile porous substrate comprising a photocatalyst connected to and extending from the mounting body; and
at least one ultraviolet lamp connected to and extending from the mounting body adjacent to the low-profile porous substrate;
positioning the low-profile porous substrate within an air flow path of the air handling system; and
activating the photocatalyst with the at least one ultraviolet lamp.

16. The method of claim 15, further comprising:
providing the at least one ultraviolet lamp as at least one elongated ultraviolet lamp and providing the low-profile porous substrate as substantially flat with a planar surface; and
positioning the planar surface and the at least one elongated ultraviolet lamp approximately perpendicular with the flow path of the of the air handling system.

17. The method of claim 16, further comprising:
inserting the at least one elongated ultraviolet lamp and the low profile porous substrate through an aperture of at least one of a duct and a plenum of the air handling system, and
connecting the mounting body to the at least one of the duct and the plenum.

18. The method of claim 17, further comprising providing the mounting body with a mounting plate and attaching the mounting plate to a surface of the at least one of the duct and the plenum with at least one fastener.

19. The method of claim 15, further comprising positioning the air purifying device in a section of the flow path wherein an area of the low-profile substrate comprises at least approximately 25 percent of the flow area of the flow path in the section.

20. A method of purifying air within an air handling system, the method comprising:
providing a low-profile porous substrate comprising a photocatalyst;
providing at least one ultraviolet lamp for activating the photocatalyst;
inserting the low-profile porous substrate approximately perpendicular to a flow path of the air handling system;
inserting the at least one ultraviolet lamp adjacent to the low-profile porous substrate within the flow path of the air handling system; and
irradiating the low-profile porous substrate with the at least one ultraviolet lamp to activate the photocatalyst.

21. The method of claim 20, further comprising:
providing the at least one ultraviolet lamp as a plurality of ultraviolet lamps;
inserting the plurality of ultraviolet lamps upstream from the low-profile porous substrate within the flow path; and
irradiating an area upstream of the lamps within the flow path.

22. The method of claim 20, further comprising irradiating the substrate and an area in proximity to the substrate with a broad spectrum of radiation including a plurality of wavelengths between about 200 nm and 300 nm.

23. The method of claim 20, further comprising irradiating the substrate and an area in proximity to the substrate with radiation including wavelengths at about 185 nm and about 254 nm.

24. The method of claim 20, further comprising:
providing a radiation sensor;
sensing an intensity of radiation from the at least one ultraviolet lamp with the radiation sensor; and
providing an indication signal from the radiation sensor when a prescribed diminished light intensity is reached.
